(19) United States
(12) Patent Application Publication
(10) Pub. No.: US 2006/0233830 A1
(43) Pub. Date: Oct. 19, 2006

(54) Vaccines

(76) Inventors: Tuen-Yee Wong, Rowville (AU); Anthony Wai-Chin So, Rowville (AU); Thomas Sai-Ying Ko, Rowville (AU)

Correspondence Address: SUGHRUE MION, PLLC, 2100 PENNSYLVANIA AVENUE, N.W., SUITE 800, WASHINGTON, DC, 20037, US

(21) Appl. No.: 10/528,364

(22) PCT Filed: Sep. 23, 2003
(86) PCT No.: PCT/AU03/01250

§ 371 Date: Jun. 8, 2006

(30) Foreign Application Priority Data
Sep. 23, 2002 (AU) 2002951692

(51) Int. Cl. A61K 39/12; A61K 39/02

(52) U.S. Cl. 424/204.1; 424/234.1; 977/802

(57) ABSTRACT

Processes for the production of a stabilised vaccine composition of labile immunogens, wherein a fluid comprising one or more immunogens is sprayed into a reactor containing fluidised particles of a pharmaceutically acceptable water soluble material at a temperature of about 25° C. to about 50° C., such that the immunogen coats and is dried onto the particles under the fluidising conditions, and thereafter collecting from said reactor dried immunogen containing particles having a moisture content between about 0.1% w/w to about 10% w/w are described. Also described are stabilised vaccine compositions of labile immunogens.

### FIELD OF THE INVENTION

The present invention generally relates to improvements in the production of vaccines, and vaccine compositions stabilised against inactivation.

### BACKGROUND

Vaccines comprising viral particles or bacterial cells or proteinaceous antigens produced by recombinant DNA technology are widely used to prevent disease in humans and animals as well as in aquaculture. Generally, viral particles and bacteria for use in vaccines are attenuated or otherwise treated with one or more agents so as to lessen or remove their pathogenicity. Genetic manipulations may be carried out to produce virus or bacteria of low or absent pathogenicity.

Vaccines have also been developed for micoplasma mediated diseases, as well as disease mediated through other infectious agents, including for example metazoans an protozoans.

It is well known that biological materials, including biological materials in solution, are susceptible to inactivation due to heat, oxidising reagents, salts, etc. Virus particles, bacteria and other infective agents used in vaccines may be readily inactivated after a short period at ambient temperature. Inactivation may result in loss of infectivity, compromised infectivity of live vaccines or at low temperature storage, or loss of immunogenicity. Many virus particles, for example human influenza virus, human hepatitis viruses, an avian bronchitis virus, may only survive at temperatures at 4° C. for a short period of time.

Vaccines, for example containing viruses used to immunise humans or animals against disease, generally require storage at temperatures of 4° C. or less, such as −20° C. Such vaccines may only be stable for a relatively short period of time, such as 2 to 14 days after production. The requirement for low storage creates handling and transport problems. Low temperature storage is costly. Short periods of vaccine activity even at low temperature limit vaccine use and raise vaccine cost and limits vaccine distribution particularly in undeveloped third world countries

Many commercially available vaccines, used for example in human and animal health, are whole particle or cell-based products in order to provide maximum protective properties following vaccine administration. Such vaccines may be live vaccines of attenuated or absent pathogenicity. These products have strict refrigeration requirements as mentioned above, and accordingly a short shelf life.

Freeze-drying under vacuum (lyophilisation) has been proposed to prepare vaccines. For example, EP-A-290197 describes freeze-dried tetravalent vaccines.

Freeze-drying processes traditionally involve freezing a solution containing an immunogen, such as virus particles, bacterial cells, or proteinaceous antigens therefrom, and converting ice crystals into water vapour under vacuum (sublimation). Unfortunately, such processes can damage the native structure of proteins, disrupt viral particles of bacterial cells. Thus a detrimental effect on the immunogen may result, this compromising or destroying immunogenicity.

Freeze-drying is also a complex process with a number of variables, and may be difficult to perform in a reproducible manner.

Another problem with freeze drying in the field of vaccine production is processing requirements. It is not possible to concentrate high doses of vaccine material in small volume. Indeed, it is important in the freeze-drying process that a large surface area of fluid is available to be in contact with the vacuum. As only the top of a frozen volume of material is in contact with vacuum, vaccines are required to be freeze-dried in large containers providing maximum surface area for freeze-drying. The necessary apparatus for such processes is therefore space consuming and inefficient. As a consequence freeze-dried vaccines are generally expensive to produce.

U.S. Pat. No. 5,616,329 describes a process where an aerosol of a microbial suspension is exposed to elevated temperature such that only heat stable components of the microbial suspension retain their immunogenic properties. The heat inactivation step according to U.S. Pat. No. 5,616,329 employs temperatures in the range of 100 to 160° C. The immunogenicity of heat labile components is lost according to this proposal, and thus this process is unacceptable in many vaccine applications.

This invention addresses various problems in the field of vaccines, including cost and production difficulties, handling and storage limitations, and vaccine stability and maintenance of immunogenicity.

### SUMMARY OF THE INVENTION

The invention disclosed herein provides in one aspect processes for the production of vaccines comprising one or more immunogens, such as viral particles, bacterial cells, micoplasmas, prions or other disease causing agents in humans, animals or aquatic species, antigenic products of disease causing organisms such as virus or bacteria, and nucleic acid sequences. The process of the invention stabilises immunogens from inactivation and loss of immunogenicity.

In accordance with the broadest process aspect of this invention there is provided a process for the production of a stabilised vaccine composition of labile immunogens, wherein a fluid comprising one or more immunogens is sprayed into a reactor containing fluidised particles of a pharmaceutically acceptable water soluble material at a temperature of about 25° C. to about 50° C., preferably about from 30° C. to 46° C., such that the immunogen coats and is dried onto the particles under the fluidising conditions, and thereafter collecting from said reactor dried immunogen containing particles having a moisture content between about 0.1% w/w to about 10% w/w so as to give a stabilised vaccine composition.

In accordance with another aspect of the invention there is provided a stabilised vaccine composition, preferably stable at ambient temperature, comprising immunogen coated particles of a pharmaceutically acceptable water soluble material, the composition having a moisture content between about 0.1% w/w to about 10% w/w.

Preferably the immunogen comprises virus particles, bacterial cells, other microorganisms, eukaryotic cells, or antigenic products thereof. The immunogen may contain two or more different virus particles, bacteria cells, other microorganisms, or antigenic products thereof etc. so as to give a multivalent vaccine composition.

Preferably the virus particles include one or more of human and animal viruses.

Examples of human viruses include: Hepatitis A virus, hepatitis B virus, hepatitis C virus; herpes simplex virus type 1 and type 2; Varicella-Zoster virus; cytomegalovirus; Epstein-Barr virus; human herpesvirus 6 and human herpesvirus 7; influenza virus; respiratory syncital virus; parainfluenza virus; adenovirus and rhinoviruses; human immunodeficiency virus and lentiviruses; human papillomavirus; measles virus; mumps virus; polio virus; rubella virus; human rotavirus; pox virus (such as smallpox virus); arbovirus transmitted disease such as Japanese Encephalitis; tick-borne encephalitis and rabies virus; yellow fever virus; West Nile virus; and dengue virus.

Examples of avian viruses include chicken influenza virus, Newcastle disease virus, avian rhino tracheitis virus, avian herpes virus, fowl pox virus, avian encephalomyelitis, infectious bronchitis, Infectious Bursal disease (Gumboro), Marek's disease virus, avian reoviris, fowl laryngotracheitis, Egg Drop Syndrome virus.

Examples of porcine viruses include Porcine Reproductive and Respiratory Syndrome, foot and mouth disease virus, porcine influenza virus, porcine parvovirus, pseudorabies virus, and porcine rotavirus, swine influenza virus.

Examples of feline viruses include feline herpes virus, feline immunodeficiency virus, feline leukemia virus, feline panleukopenia, feline viral rhinotracheitis, feline calicivirus, feline viral rhinotracheitis, feline coronavirus and rabies.

Examples of canine viruses include canine distemper virus, canine adenovirus, parainfluenza, and canine parvovirus, canine hepatitis virus canine herpesvirus and rabies.

Examples of equine viruses include equine encephalitis virus (Eastern, Western and Venezuelan equine viral encephalomyelitis), equine influenza, and equine herpesvirus (equine rhinopneumonitis).

Examples of bovine viruses including infectious bovine rhinotracheitis, bovine virus diarrhea virus bovine respiratory syncytial virus, coronavirus, foot and mouth disease virus and parainfluenza.

Preferably, virus particles are live. As vaccine compositions according to this invention are free flowing powders, vaccines containing different immunogens can be simply blended together free of compatibility problems which may otherwise arise with conventional liquid vaccine admixtures. Thus a preferable aspect of this invention is multivalent vaccine compositions containing two or more different vaccine compositions.

Preferably the bacterial cells comprise one or more bacteria from bacterial genus, including: Escherichia, such as Escherichia coli including enterotoxigenic, enteropathogenic, enteroinvasive and enteroaggragative E. coli; Salmonella, such as Salmonella Typhi and Salmonella enteritidis; Haemophilus, such as Haemophilus influenzae including H. influenza Serotype B, Haemophilis parasuis Heamophilis somnus and Haemophilis paragallinarum (Infectious Coryza); Chlamydia, such as Chlamydia pneumoniae and Chlamydia trachomatis; Neisseria, such as Neisseria meningitidis; Vibrio, such as Vibrio cholerae; Group A and Group B Streptococcus, such as Streptococcus pneumoniae (pneumococcus) and Streptococcus suis; Legionella, such as Legionella pneumophila; Bacillus, such as Bacillus anthracis; Mycobacterium, such as Mycobacterium leprae and Mycobacterium paratuberculosis; Clostridium, such as Clostridium bolutinum, Clostridium tetani, Clostridium prefringengens and Clostridiun Difficile; Pasteurella such as Pasteurella multocida and Pasteurella heamolytica; Bordetella such as Bordetella bronchiseptica and Bordetella pertussis; Actinobacillus such as Actinobacillus pleuropneumoniae and Actinobacillus suis; and bacteria including Erysipelothrix rhusiopathiae; Leptospira; Borrelia burgdorferi; Helicobacter pylor; and Corynebacterium diphtheriae.

Mycoplasma such as Mycoplasma hyopneumoniae, Mycoplasma gallisepticum, Mycoplasma synoviae and Mycoplasma pneumoniae may be used in this invention.

Preferably the immunogen comprises antigenic products from disease causing viruses, bacteria, and/or other disease causing microorganisms. Such antigenic products include viral sub-particles, viral particles without their nucleic acid content, viral proteins, bacterial proteins, bacterial lipopolysaccharides, glycoproteins, carbohydrates or two or more of the aforementioned antigenic products. Antigenic products may be epitopes comprising a sequence of amino acids, or polysaccharides, antigens produced by recombinant DNA technology or the like, derived from viral and/or bacterial proteins and/or carbohydrate and/or lipid sequences, optionally conjugated to a carrier, such as a peptide or protein.

Preferably the vaccine composition is a free flowing particulate composition.

Preferably the immunogen coating of the pharmaceutically acceptable water soluble material includes additional constituents such as amino acids, proteins, chelating agents, buffers, preservatives, stabilisers, antioxidants, emulsifiers, plasticizer and lubricants.

Preferably the immunogen coating includes an adjuvant such as aluminium salts (alum), muramyl peptides and analogues or derivatives, saponins (for example Quillaja saponin) or saponin containing compounds (for example ISCOM®), polynucleotides or synthetic nucleic acid derivatives such as polyribonucleotides, sulfur-containing compounds such as Levamisole, polymers and heterocyclic and aromatic compounds such as Divema and pluronic polyols, amine and lipid-containing compounds, avridine, dimethyldoctadecyl-ammonium bromide, polyphosphaze, cytokines (such as interferon) or biodegradable water in oil emulsions such as emulsified paraffin. Other adjuvants or agents with immunostimulation or immunomodulating or antigen presenting properties, and commercial products Impran, Emunade, Emulsigen, and/or Amphigen may also be used.

Preferably the vaccine composition is a live vaccine, that is, immunogens are capable of inducing immune responses by mimicking natural viral infection in an immunized subject. Live vaccine compositions may be stable for periods up to 30 days or more storage at 4° C. and at ambient temperatures, for example at 25° C. For example, where the immunogens are virus particles, the virus particles may be live and infective at the completion of the process, yet stable at room temperature storage. Live vaccine compositions so produced may be stable for periods up to 30 days or more storage at ambient temperature, for example at 25° C.

Preferably the fluid comprising one or more immunogens contains a suspension or dispersion of immunogens, such as viral particles, bacterial cells or other microorganisms, eukaryotic cells, or antigenic products of viral particles, bacterial cells or other microorganisms.

The fluid comprising one or more immunogens is may be a culture medium or other aqueous fluid or media containing the immunogen. The fluid may include one or more additional constituents such as amino acids, proteins, chelating agents, buffers, salts, preservatives, stabilisers, antioxidants, emulsifiers, plasticizer and lubricants.

### DESCRIPTION OF THE FIGURE

FIG. 1 is a bar graph plotting decrease in viral activity (from initial activity) measured in logEID₅₀ values for live avian vaccine compositions. ND-VB refers to a stabilised Newcastle Disease vaccine according to the present invention. ND-FD refers to a freeze dried Newcastle Disease vaccine. IB H120-VB refers to an Infectious Bronchitis vaccine produced according to the present invention. IB H120-FD refers to a freeze dried Infectious Bronchitis vaccine. IBD-VB refers to a stabilised Infectious Bursal Disease vaccine produced according to the present invention. IBD-FD refers to a freeze dried Infectious Bursal Disease vaccine. The samples were stored at 25° C. for 30days.

### DETAILED DESCRIPTION

This invention in its various embodiments provides processes for the production of vaccine compositions and stabilised vaccine compositions. The processes of the invention are suitable for the production of vaccines containing viral particles, bacterial cells, mycoplasmas, prions other disease causing agents in humans, animals or aquatic species, or antigenic products or viral particles, bacterial cells, mycoplasmas, prions or other disease causing agents. The process of the invention provides, in one preferred embodiment, high potency live vaccines, stable at room temperature for extended periods, and processes for their production. Such vaccine compositions have hitherto been unknown.

In accordance with one aspect of this invention there is provided a process for the production of vaccines comprising one or more immunogens, such as viral particles, bacterial cells, mycoplasmas, prions or other disease causing agents in humans, animals or aquatic species, or antigenic products thereof. The process of the invention stabilises immunogens from inactivation and loss of immunogenicity. In this aspect the invention provides a process for the production of a stabilised vaccine composition of immunogens, particularly labile immunogens, wherein a fluid comprising one or more immunogens is sprayed into a reactor containing fluidised particles of a pharmaceutically acceptable water soluble material at a temperature of about 25° C. to about 50° C., preferably about 30° C., such that the immunogen coats and is dried onto the particles under the fluidising conditions, and thereafter collecting from said reactor dried immunogen containing particles having a moisture content between about 0.1% w/w to about 10% w/w so as to give a stabilised vaccine composition.

The immunogen may comprise virus particles, bacterial cells, other microorganisms or eukaryotic cells or antigenic products thereof. The immunogen may contain two or more different virus particles, bacterial cells, other microorganisms or antigenic products thereof so as to give multivalent vaccines.

Any type of virus particle, or bacterial cell, mycoplasma, prion or other disease causing agents in humans, animals or aquatic species, or antigenic products of virus particles, bacterial cells, mycoplasmas, prions or other disease causing agents may be used in this invention.

Preferably the immunogen comprises virus particles. Preferred virus particles include: hepatitis A virus, hepatitis B virus, hepatitis C virus; herpes simplex virus type 1 and type 2; Varicella-Zoster virus; cytomegalovirus; Epstein-Barr virus; human herpesvirus 6 and human herpesvirus 7; influenza virus; respiratory syncital virus; parainfluenza virus; adenovirus and rhinoviruses; human immunodeficiency virus and lentiviruses; human papillomavirus; measles virus; mumps virus; polio virus; rubella virus; human rotavirus; pox virus (such as smallpox virus); arbovirus transmitted disease such as Japanese Encephalitis; tick-borne encephalitis and rabies virus; yellow fever virus; West Nile virus; dengue virus; avian viruses; porcine viruses; feline viruses; canine viruses; equine viruses; and bovine viruses.

Preferably, the bacterial cells comprise one or more bacteria from bacterial genus including: Escherichia, such as Escherichia coli including enterotoxigenic, enteropathogenic, enteroinvasive and enteroaggragative E. coli; Salmonella, such as Salmonella Typhi and Salmonella enteritidis; Haemophilus, such as Haemophilus influenzae including H. influenza Serotype B, Haemophilis parasuis Heamophilis somnus and Haemophilis paragallinarum (Infectious Coryza); Chlamydia, such as Chlamydia pneumoniae and Chlamydia trachomatis; Neisseria, such as Neisseria meningitidis; Vibrio, such as Vibrio cholerae; Group A and Group B Streptococcus, such as Streptococcus pneumoniae (pneumococcus) and Streptococcus suis; Legionella, such as Legionella pneumophila; Bacillus, such as Bacillus anthracis; Mycobacterium, such as Mycobacterium leprae and Mycobacterium paratuberculosis; Clostridium, such as Clostridium bolutinum, Clostridium tetani, Clostridium prefringengens and Clostridiun Difficile; Pasteurella such as Pasteurella multocida and Pasteurella heamolytica; Bordetella such as Bordetella bronchiseptica and Bordetella pertussis; Actinobacillus such as Actinobacillus pleuropneumoniae and Actinobacillus suis; and bacteria including Erysipelothrix rhusiopathiae; Leptospira; Borrelia burgdorferi; Helicobacter pylor; and Corynebacteium diphtheriae.

Mycoplasma such as Mycoplasma hyopneumoniae, Mycoplasma gallisepticum, Mycoplasma synoviae and Mycoplasma pneumoniae may be used in this invention

Virus particles, bacterial cells, other microorganisms or still other immunogens may be alive or intact, that is not killed by heat treatment or other processes. The processes according to a preferred embodiment of this invention are adapted for the production of vaccine compositions containing live immunogens, such as viral particles. On administration to a subject, whether human, animal or aquatic species, the vaccine compositions containing live virus particles or other immunogens elicit a strong immune reaction.

Alternatively, viral particles, bacterial cells, other microorganisms, or still other immunogens, may be killed, that is heat treated or otherwise treated such that they are not capable of reproduction in a host. Subunits or other antigenic products, for example of virus particles or bacterial cell fractions, may also be used in the invention, as may antigens thereof, such as peptides, proteins, carbohydrates, lipids, lipopolysaccharides, glycoproteins, or two or more of the aforementioned antigenic products. Antigenic products may be epitopes comprising a sequence of amino acids, or polysaccharides, or the like, derived from viral and/or bacterial proteins and/or carbohydrate and/or lipid sequences, optionally conjugated to a carrier, such as a peptide or protein.

In an alternative embodiment the immunogen may be a nucleic acid sequences, such as DNA or RNA, for example based on viral or bacterial or other microorganism nucleic acid sequences, which may, for example, be delivered in viral vectors such as pig or fowl adenovirus or fowl pox virus or other viral vectors which are stabilised according to the present invention.

In the process aspect of this invention, one or more immunogens are provided in a fluid. The fluid preferably comprises a suspension or dispersion of immunogens, such as viral particles bacterial cells or other microorganisms or eukaryotic cells. The fluid may be a culture medium or other fluid or media containing the immunogen, optionally diluted with a diluent in which the immunogen is stable (that is, not inactivated). Diluents are well known in the art of virology and microbiology, and include, for example, sterile water, phosphate buffered saline (PBS), tris buffered saline (TBS), sterile water containing sucrose and skim milk (an example being 5% of both sucrose and skim milk, and 90% sterile water). Diluents may also include one or more of gelatin, dextran, EDTA, amino acids such as glycine and egg albumin, mineral salts such as magnesium sulphate, calcium chloride, and calcium phosphate, and the like.

In an advantageous aspect of this invention, commercially available vaccines, whether for human, animal, avian or other species use, may be stabilised against inactivation, for example on storage at ambient temperature. In this regard, commercially available live vaccines, or different types of virus particles, or bacterial cells, or different types of bacterial cells, may be diluted with a diluent in which the virus or bacteria is stable so as to give a fluid comprising one or more immunogens suitable for stabilisation according to the process of this invention.

Globally, the market for human vaccines has been estimated in 2001 to be a US$5-6 billion market. The majority of live viral or bacterial vaccines for use in human health are attenuated, such as being non-pathogenic strains, or strains of limited pathogenicity, for example produced by recombinant DNA technology or other means. Other commercially available vaccines include viral or bacterial proteins, or proteins/peptides derived therefrom, such as epitope vaccines containing peptide, protein, glycoprotein, or other epitopes of disease causing virus, bacteria, or other organisms, optionally associated with a carrier such as a further peptide, protein or other agent(s), for example by covalent bonding or other association.

Applicants believe that any commercially available vaccine may be stabilised in accordance with this invention. For example, a commercially available vaccine may be diluted with an appropriate diluent in which the vaccinating organisms, such as one or more different viral particles, or one or more different bacterial cells are stable, to give an immunogen containing fluid. The immunogen containing fluid may then be sprayed into a reactor containing fluidised particles in accordance with the process of this invention. Examples of vaccines which may be used in the present invention include those from the following manufacturers:

### Human Vaccines

Aventis Pasteur

Acellular pertussis and/or Hib paediatric combinations with product names including Tripedia/Tripacel, Quadracel/Tetravac, Tetract-Hob, Pentact-Hob/Pentacel/Pentavac, Hexavac
TyphinVi/Menomune/Avaxim/Venorab/Stamaril, for travelers/endemic area
Vaxirip/Fluzone/Mutagrip, for influenza
GenHevac B Pasteur, for hepatitis B
IPOL/Imovax Polio, injectable polio vaccine
Tetanus/Diptheria vaccines
   GlaxoSmithKline
Havrix, for hepatitis A
Engerix-B, for hepatitis B
Twinrix, for hepatitis A and B (adult and paediatric)
Infanrix, paediatric diphtheria/tetanus/pertussis
Infanrix PeNta, for paediatric hepatitis B/Polio
Infan HeXa, for paediatric haemophilus influenza type B (Hib)
Priorix, for measles/mumps/rubella
Typherix, for typhoid fever
Varilirix, for varicella (chicken pox)
Simplirix, for herpes simplex virus (type 2) (genital herpes) (under development)
Dengue fever vaccine (under development)
Epstein-Barr virus vaccine (under development)
Human papillomavirus vaccine (under development)
Meningitis B (Cuba) vaccine (Meningococcal B) (under development)
Streptococcus pneumoniae vaccine (under development)
Neisseria meningitidis vaccine (under development)
Hepatitis C vaccine (under development)
   Wyeth
Pnu-Imune 23, Pneumococcal vaccine, Polyvalent, for meningitis and blood infections
Prevnar, Pneumococcal 7-valent conjugate vaccine (Diphtheria CRM₁₉₇ protein conjugate). Differs from other marketed pneumococcal vaccines with the ability to induce immunity in children under two years, who are susceptible to invasive pneumococcal disease.
HibTiter, haemophilus b Conjugate vaccine (diphtheria CRM₁₉₇ protein conjugate) for paediatric haemophilus influenza type B
FluShield, influenza virus vaccine, trivalent, types A and B (purified subviron)
Meningitec, for meningococcocal group C
   Merck
Vaqta, inactivated vaccine against hepatitis A
Meruvax II, live vaccine against rubella
M-M-R II, live vaccine against measles, mumps and rubella
Varivax, live vaccine against varicella
Recombivax HB, recombinant vaccine against hepatitis B
Pedvax HIB, haemophilus b conjugate vaccine (mengingococcal protein conjugate)
Comvax, vaccine against haemophilus b conjugate and hepatitis B
Pneumovax 23, pneumonococcal vaccine, polyvalent
Human papillomavirus vaccine for cervical cancer and genital warts (under development)
Rotavirus vaccine (under development)
   Chiron (Powderject)
Menjugate, conjugated vaccine against meningococcal C disease
Fluad, adjuvanted influenza vaccine
Begrivac, the first preservative-free influenza vaccine
Encepur, vaccine against tick-borne encephalitis
vaccines against polio, rabies, paediatric diphtheria/tetanus/pertussis, measles/mumps/rubella
Fluvirin, for influenza,
Dukoral, for travel diarrhea and cholera (oral vaccine)
Arilvax, for yellow fever
BCG vaccine for tuberculosis
Nathav, inactivated vaccine against Hepatitis A
Meningococcal B (New Zealand) vaccine (under development)
Meningococcal multivalent (ACYW) vaccine (under development)
Hepatitis C vaccine (under development)
   Baxter
meningococcal C conjugate vaccine
influenza vaccine (under development)
tick-borne encephalitis vaccine

Veterinary vaccines may also be utilised in accordance with this invention. Commercially available vaccines include, by manufacturer:

Merial

Nemovac to prevent avain rhinotracheitis
Gallimune, range of inactivated vaccines
four live and six inactivated vaccines including Lyomarex, Avinew, Dur706, Bioral H 120 and Haemovax (inactive)—used against most common poultry diseases which are marketed by Glaxo India
LT-Blan, for laryngotracheitis caused by an avian herpes
Hyoresp (inactivated vaccine) for active immunization against infection and lung lesion of disease caused by Mycoplasma hyopneumoniae
Parvoruvax/Parvovax/Parvoject/Ruvax for active immunization against porcine parvovirus and swine erysipelas
Neocolipor (containing inactivated strains of E-coli) for reducing neonatal enterotoxicosis in piglets
FMD, inactivated and adjuvanted Foot and Mouth Disease virus
   Intervet

Poultry Vaccine Products

Live Vaccines

Products for preventing Newcastle Disease, infectious bronchitis, coccidiosis, fowl pox, fowl cholera, reovirus induced tenosynovitis (viral arthritis), fowl laryngotracheitis, avian encephalomyelitis, infectious bursal disease (IBD), Marek's Disease and mycoplasma gallisepticum infection. Products variants include Reo ST 1133, AE Pox, Gumboro, Rismavac, Lasota, Clone 30, H120, IB MA5, ILT OVO-Diptherin, MG6/85.

SG9R, live freeze-dried vaccine against Salmonella gallinarum and Salmonella enteriditis infections in chickens

Inactivated Vaccines

Products for Newcastle Disease, Coryza Disease, Egg Drop syndrome, infectious bronchitis, mycoplasma gellisepticum infection and reovirus induced tenosynovitis (viral arthritis). Products variants include Newcavac, Coryza, EDS 76, IB+ND, REO IB+G+ND, REO INAC, MG INAC.

Pig Vaccines Products

A range of 15 inactivated and freeze dried live attenuated vaccine products, including combination formulations, against Actinobacillus pleuropneumoniae, atrophic rhinitis, pseudorabies, swine erysipelas, parvovirus, E-coli enterotoxicosis, myoplasma hyopneumoniae. Product name are Porcilis with the strain or ProSystem. Tentanus serum is also marketed.

Pfizer

Stellamune Once/RespiSure-ONE for mycoplasma hyopneumoniae in pigs. This once dosage product is the largest selling pig vaccine (300 millon doses globally)
RespiSurePleuroguard-4, for influenza in pigs
ER Bac/ER Bac Plac, for erysipelas
FarrowSure/FarrowSure B/FarrowSure B-PRV/FarrowSure PRV, for porcine parovirus, leptospira strains, erysipelas and pseudorabies
PR_Vas Plus/PR-Vac, for pseudorabies
LiterGuard/LitterGuard LT/LitterGaurd LT-C, for enterotoxicosis caused by E-coli and Clostriduim perfringens type C. The LT variant contains heat-labile toxiod (LTb) stimulates protection against the enterotoxins that E-coli produce
Poultry vaccine against coccidosis
   Wyeth/Fort Dodge Animal Health

Poultry vaccine products
Bursine-2, live vaccine for protection of infectious bursal disease in chickens
Poulvac H120/Poulvac IBMM/IB Primer/IBMM+ARK, for infectious bronchitis in poultry
AE Vac/AE Poxine, modified live vaccine for avian encephalomyelitis, fowl pox
LT Vac, live vaccine for fowl infectious laryngotracheitis
MD Vac Lyo, freeze dried live turkey herpes virus vaccine against Marek's Disease
ND Hitchner/ND La Sota, freeze dried modified vaccine against Newcastle Disease
VA Chick Vac/VA Vac, freeze dried, modified live vaccine against tenosynovitis (viral arthritis)

Range of inactivated vaccines for Infectious Bursal Disease, Newcastle Disease, Infectious Bronchitis, Egg Drop Syndrome, Infectious Coryza (Haemophilis pasagallinarum), Mycoplasma gallisepticum, Mycoplasma synoviae and avian reovirus. Product names include Bursine K, Chick NK, Coryza Vac, EDS New Bronz, EDS Vac, MG Bac, MS Bac, New Bronz, New Bronz MG, Newcastle K, Coryza Oil 3, Provac 3, Provac 4 Tri Reo.

Pig Vaccine Product
Suvaxyn Respifend/RespiFend MH, an inactivated and adjuvanted bactein for protection against mycoplasma hyopneumoniae in pigs
Suvaxyn EC4/Maternafend 4, E coli
Suvaxyn P/Gestfend 1, parvovirus
Suvaxyn L/Gestafend 5, 5 strains of leptospira
Suvaxyn/Gestafend 5+B, leptospira with bratislava
Suvaxyn E/Herdfend Thrix, Erysipelothrix for swine erysipelas
Suvaxyn PL/Suvaxyn PLE/Suvaxyn PLE+B/Gestafend 6, Gestafend 7, Gestafend 7+B, combination vaccines of parvovirus, leptospira, and erysipelothrix
Suvaxyn AR/T/E, Bordetella bronchiseptica, Pasteurella multocida, Erysipelothrix for atrophic rhinitis, pneumonia and erysipelas.
   Boehrinzer Ingelheim

Pig Vaccine Products
Ingelvac PRRS MLV, first modified live vaccine against Porcine Reproductive and Respiratory Syndrome (PRRS)
Ingelvac DART, to protect against toxigenic strains of Bordetella and multocida types A and D
Ingelvac M.hyo, one dose, 120 day vaccine for the prevention of pneumonia cause by Myoplasma hyopneumoniae
Ingelvac-HPE, vaccine against Haemophilus parasuis and erysipelas
Ingelvac Aujeszky MLV, modified live vaccine against Aujeszsky Disease (pseudorabies)
ReproCyc PRRS-PLE, combination vaccine against PRRS, parvovirus, leptospira, and erysipelas
Ingelvac PRRS ATP, highly attenuated, modified live vaccine against atypical PRRS
Ingelvac PRRS HP/Ingelvac PRRS HPE, combination vaccines against PRRS and Haemophilus parasuis and erysipelas
Ingelvac AR4, vaccine against atrophic rhinitis caused by Bordetella bronchiseptica and Pasteurella multocida type D

Poultry Vaccine Products

Range of mono- and polyvalent, modified live and attenuated vaccine for Newcastle, Bronchitis and coryza various strains and variants under the trade name Volvac.

Schering Plough

Pig Vaccine Products
Scourmune/Scourmune-C/Scourmune-CR, E-coli (only vaccine that protects against Type I pili), and combination products with Clostridium perfringens and Rotavirus with group A serotypes 4&5 for diarrhea
SS Pac, Streptococcus suis for prevention of meningitis, arthritis, pneumonia and septicemia.
Parapac, Haemophilus parasuis for prevention of Glasser's Disease
Pneu Pac/Pneu Parapac+ER/Pneu Pac-ER, Actinobacillus pleuropneumoniae serotypes 1,5&7 product and combination with Erysipelothrix rhusiopathiae or Haemophilus parasuis for prevention of pneumonia and erysipelas/Glasser's disease.
AR-Pac-PD+ER/AR-Parapac+ER, Bordetella bronchiseptica with Erysipelothrix rhusiopathiae and Pasteurella multocida or combination with Haemophilus parasuis for prevention of atrophic rhinitis, pneumonia, erysipelas, Glasser's Disease
M+Pac, mycoplasma hyopneumoniae for protection against of pneumonia in pigs
MaxiVac-Flu, killed virus Type A H1N1 subtype against influenza in pigs
PRV-Marker Gold/PRV-Marker Gold-MaxiVac Flu, for pseudorabies and swine influenza with modified and killed virus
Prime Pac PRRSV, modified live virus against influenza in pigs

Poultry Vaccine Products

Live and modified live vaccine products for preventing Newcastle Disease, Infectious Bronchitis (various strains), coccidiosis, fowl pox, fowl cholera, reovirus induced tenosynovitis (viral arthritis), fowl laryngotracheitis, avian encephalomyelitis, Infectious Bursal Disease (IBD) and mycoplasma gellisepticum infection. Product names include Shor-Bron-D, Ava-Bron, Broilerbron, Coccivac, Paracox, Monovax, Twin Vax, Polybron, Avichol, Enterovax, F Vax-MG, LT-Ivax, M-Ninevax, Ocuvax, Polyvax-TC, Trachivax, Univax, Variant vax-BD, PM-Onevax-C, Burs-Vac, Teno-Vaxin, Broilertrake, Ava-Trem, Ava-Pox.

Bioproperties Australia

Live vaccines
Vaxsafe MG, Mycoplasma gallisepticum for control of CRD in poulty
Vaxsafe MS, Mycoplasma synoviae for post antibiotic problems in chickens
Salvax, salmonella typhimuium for control of most salmonella sp. in poultry
Mareks HVT, herpes virus of turkeys for chickens
Mareks Rispens, CVI 988 strain of Marek's disease virus
Eimeriavax 4, four Eimeria strains precocious vaccine for control of coccidiosis in chickens
Vaxsafe IBD, Infections Bursal Disease for chickens
Vaxsafe IB, Infectious Bronchitis virus for poultry
Vaxsafe PM, Pasteurella multocida for fowl cholera in poultry
Vaxsafe MH, Mycoplasma hyopneumoniae

Vaccine trade marks are generally shown in italics above.

The vaccine manufacturers referred to above are generally multinational corporations operating in many countries of the world.

The aforementioned vaccine compositions, or the immunogens contained in them, may be used in this invention in one embodiment. Further, vaccines against one or more of the human or animal diseases/infectious agents referred to above form a further embodiment of this invention.

In accordance with another aspect of this invention there is provided an ambient temperature stable vaccine composition comprising immunogen coated particles of a pharmaceutically acceptable water soluble material, the composition having a moisture content between about 0.1% w/w to about 10% w/w. The vaccine composition may be produced according to the process of this invention as herein described.

In the process aspect of this invention, fluid comprising one or more immunogens is sprayed into a reactor containing fluidised particles of a pharmaceutically acceptable water soluble material at a temperature from about 25° C. to about 50° C., preferably about 30° C. to about 46° C. such that the immunogen coats and is dried on to the particles under the fluidising conditions, and thereafter dried immunogen containing particles having a moisture content between about 0.1% w/w to about 10% w/w are collected, giving a stabilised vaccine composition.

Fluid comprising one or more immunogens is preferably a suspension or dispersion of immunogens, such as viral particles, bacterial cells or other microorganisms, or eukaryotic cells. The fluid may be a culture medium in which, for example, virus particles are propagated or stored in stock. The fluid may, for example, be a culture medium or other fluid media containing the immunogen. For example, conventional components used in the freeze drying of bacteria and/or virus particles may be used, as are well known in the art. Examples include a mixture of sucrose, skim milk and sterile water, or phosphate buffered media at around pH 7, containing for example disodium EDTA, egg albumin, and glycine. The immunogen containing fluid may include one or more of amino acids, proteins, chelating agents, buffers, preservatives, stabilisers, metal antioxidants and lubricants.

Preferably the immunogen coating includes an adjuvant such as aluminium salts (alum), muramyl peptides and analogues or derivatives, saponins (for example Quillaja saponin) or saponin containing compounds (for example ISCOM®), polynucleotides or synthetic nucleic acid derivatives such as polyribonucleotides, sulfur-containing compounds such as Levamisole, polymers and heterocyclic and aromatic compounds such as Divema and pluronic polyols, amine and lipid-containing compounds, avridine, dimethyldoctadecyl-ammonium bromide, polyphosphaze, cytokines (such as interferon) or biodegradable water in oil emulsions such as emulsified paraffin. Other adjuvants or agents with immunostimulation or immunomodulating or antigen presenting properties, and commercial products Impran, Emunade, Emulsigen, and/or Amphigen may also be used.

Any pharmaceutically acceptable water soluble material or mixture of materials may be utilised in the invention. By “water soluble” is meant 1 g of material dissolves in 1 ml to 10 ml of water at 20° C. The pharmaceutically acceptable water soluble material may comprise one or more monosaccharides, disaccharides, polysaccharides or carbohydrates. Examples include dextrose, mannitol, fructose, polyfructosan, polydextrose, dextrin, glucose, invert sugar, lactitol, lactose, isomalt, maltitol, maltose, maltodextrin, sorbitol, xylitol, sucrose, sucralose, mannose, galactose, xylose, arabinose, fructose, glucosamine, galactosamine, rhamnose, 6-O-methyl-D-galactose, 2-0-acetol-beta-D-xylose, 2-acetamido-2-dioxy-beta-D-galactose-4-sulphate, N-acetylglucosamine, iduronate, mannuronate, methyl galacturonate, galactose, arabinose, alpha-D-manopyranose and biopolymers formed by covalent bonding between one or more monosaccharide or disaccharide units. Examples of carbohydrates include alginate, amylose, cellulose, carrageenan, pectin. For convenience, monosaccharides, disaccharides, polysaccharides and carbohydrates may be collectively referred to as “sugars”.

The pharmaceutically acceptable water soluble material may, alternatively, comprise a water soluble peptide or peptides (such as casein hydrosolate, or gelatine, or gelatine hydrosolate), mineral salts such as aluminium hydroxide, sodium chloride, sodium phosphate, sodium acid phosphate, EDTA sodium, magnesium chloride, magnesium sulphate, or a water soluble polymer. Water soluble polymers generally contain at least 10 monomer units in the polymer chain, and form an aqueous solution in water. Examples include water soluble gums, pectin, carboxymethyl cellulose, methyl cellulose hydroxyethyl methylcellulose, hydroxyethyl cellulose, hydroxypropyl methylcellulose and hydroxypropyl cellulose.

Water soluble pharmaceutically acceptable excipients, well known in the pharmaceutical/veterinary field, may in one embodiment be utilised in this invention as the pharmaceutically acceptable water soluble material. Examples of pharmaceutically acceptable excipients are provided, for example, in Martindale, The Extra Pharmacopoeia, 33rd Edition, The Pharmaceutical Press, London, 2002, which is incorporated herein by reference. Examples of pharmaceutically acceptable water soluble excipients include compressible sugar, confectioner's sugar, dextrates, potassium chloride, or, sugar spheres. Two or more excipients may be used.

Particles of a pharmaceutically acceptable water soluble material preferably have a particle size from 20 microns to 1 mm. More preferably the size of the particles is from about 50 to about 200 microns.

The process of the invention may be carried out in any spray drying reactor, or fluidised bed spraying apparatus, as are well known in the art. Examples include a PLC (Programmable Logic Controller) Driven Turbojet™ Fluid Bed Coater manufactured by BWI Huttlin (Daimlerstrasse 7, D-79585, Steinen, Germany), a PSD™ Pharmaceutical Spray Dryer from Niro, Inc (Columbia, Md. 21045 USA), and fluid bed dryers from Glatt (Ramsay, N.J. 07446, USA) or Vector-Freund (Marion, Iowa 52302, USA).

The present invention is distinct from spray drying proposals known in the art in that the immunogen containing fluid is sprayed onto fluidised particles and dried thereon. In contrast, in spray drying techniques a solution or slurry is sprayed into an air stream and dries under the fall of gravity. The process of this invention is particularly advantageous as it gives rise to vaccine compositions in a relatively short time period. For example a 2 kg batch of vaccine composition can be produced in less than an hour. Freeze drying of a similar amount of material may take 1 to 3 days or more. Moreover, the compositions of this invention are more stable than freeze-dried vaccines.

The fluid comprising one or more immunogens is preferably sprayed through a nozzle or spray head which delivers the sprayed fluid into the reactor. The fluid comprising one or more immunogens may be sprayed into the fluidised particles at any position from the base of the fluidizing zone to and the top, for example of a fluidizing bed. Spray nozzles may be embedded in a fluidised bed or otherwise located in a reactor so as to deliver a spray of the fluid comprising one or more immunogens to the fluidised particles.

It is desirable, but not essential to this invention, to utilise fluidising conditions which exceed those generally used in fluid bed operations in a reactor. Conventionally, equipment manufacturers do not recommend exceeding 50% w/v capacity of the processing chamber of fluidised particles or materials. Whilst fluidised particles may comprise from 20/50% w/v capacity of the reactor, the process in accordance with an embodiment of the present invention allows for processing weight:reactor volume to be more than 50% w/v.

In a specific, non-limiting, embodiment of the invention particles may be loaded into a reactor containing a fluidised bed, for example a spray coating apparatus which is modified to contain fluidised particles, such that fluidisation occurs, for example, at a rate between 200 to 500 m²/h.

Fluidisation is preferably conducted at a temperature between about 30° C. to about 46° C.

A desired quantity of fluid comprising one or more immunogens is sprayed onto fluidised particles of a pharmaceutically acceptable water soluble material, for example a sugar. Coating of the particles and drying of immunogen coating takes place in the fluidising conditions of the bed. Velocity of fluidisation, and flow rate of immunogen fluid into the fluidising conditions are adjustable variables which allow for the vaccine composition to be dried to a desired moisture content. The moisture content of the vaccine composition is between about 0.1% w/w to about 10% w/w, giving a stabilised vaccine composition.

It will be appreciated that reactor conditions, and flow rates of immunogen, including spraying of fluid comprising one or more immunogens into a reactor containing, for example, a fluidised bed, may be readily altered. Alterations may be made, for example, to fluidised air volume, liquid spraying speed, spray liquid temperature, humidity of inlet air, and the like. Where an alteration is made to one parameter a person of general skill in the art to which the invention relates will readily be able to identify any corresponding adjustments which may be required in another parameter to compensate for the first said alteration.

Moisture content of materials is readily measured by methods known in the art, including infrared moisture analysis such as Fourier Transfer-Near Infrared (FT/NIR) spectroscopy for example the Thermo Nicolet Antaris FT/NIR analyser from Thermo Electron Corporation, Waltham, Mass., USA), halogen heating moisture analyser (for example an MB35 or 45 moisture analyser from Ohaus Inc, Pine Brock, N.J., 07058, USA).

The stabilised vaccine composition preferably has particle size from 50 to 400 microns, more preferably 50 to 200 microns.

The process according to one aspect of this invention allows the production of a vaccine composition having a water content between about 0.1% w/w to about 10% w/w. Preferably, the water content is about 0.1% w/w to about 4% w/w, more preferably about 0.2% w/w to about 1.5% w/w. Freeze drying techniques produce relatively high moisture contents as a consequence of the freeze drying methodology. The high moisture content of freeze dried vaccines may be associated with storage difficulties and loss of activity on storage. Low moisture content vaccines, such as 0.1% w/w to about 2% w/w water content produced according to a preferred embodiment of the present invention, are particularly stable with maintenance of activity on storage, including storage at ambient temperature, such as 15-37° C., typically 25° C. for 30 days.

The stabilised vaccine composition according to an embodiment of this invention comprises immunogen coated particles of pharmaceutically acceptable water soluble material, with the composition having a moisture content between about 0.1% w/w to about 10% w/w as mentioned above. The immunogen coats the particles. The immunogen containing fluid used to coat the particles may include other components including one or more of amino acids, chelating agents, buffers, preservatives, stabilisers, mineral salts, antioxidants and lubricants.

Components present in the immunogen fluid used to form the composition of the invention generally form part of the coating of the particles, unless evaporated during drying.

Preferably the immunogen coating includes an adjuvant such as aluminium salts (alum), muramyl peptides and analogues or derivatives, saponins (for example Quillaja saponin) or saponin containing compounds (for example ISCOM™), polynucleotides or synthetic nucleic acid derivatives such as polyribonucleotides, sulfur-containing compounds such as Levamisole, polymers and heterocyclic and aromatic compounds such as Divema and pluronic polyols, amine and lipid-containing compounds, avridine, dimethyldoctadecyl-ammonium bromide, polyphosphaze, cytokines (such as interferon) or biodegradable water in oil emulsions such as emulsified paraffin. Other adjuvants or agents with immunostimulation or immunomodulating or antigen presenting properties, and commercial products Impran, Emunade, Emulsigen, and/or Amphigen may also be used.

The composition of this invention is a stabilised vaccine composition. By this is meant that the vaccine composition is not inactivated on storage for 30 days at 25° C., remaining efficacious, for example, giving rise to protective immunity. By way of further example, a viral vaccine composition is stabilised against inactivation or stable where a drop in EID 50 (50% embryo infective dose) on storage at 25° C. for 30 days is less than one log. Preferably, the composition is stable at ambient temperature, for periods of up to 30 days or more, such as from 1 to 7 days, 4 to 14 days, 7 to 30 days, or 30 to 120 days storage at ambient temperature, for example 15-35° C. The stabilised vaccine composition may be stored at 4° C. for extended periods, in contrast to traditional vaccine compositions known in the art. For example, the vaccine compositions of this invention may be stored for periods up to a year or more at 4° C. A vaccine composition stabilised against inactivation remains active in providing an immune response when administered to a subject whether human, animal, bird, fish or other subject in need of vaccination for protection from disease.

The process of the present invention, and the vaccine composition resulting therefrom, may provide in one embodiment a live vaccine where immunogens are capable of reproduction in an immunised host. For example, where the immunogens are virus particles, the virus particles may be alive and infective at the completion of the process of the invention, and infective and stabilised against inactivation in the composition aspect of this invention. Live vaccine compositions of this invention may be stabilised for periods of up to 30 days or more at ambient temperatures, for example 25° C. as mentioned above.

The vaccine composition, for example containing virus particles or bacteria, may be used as a carrier (such as a vector) for delivery of DNA or RNA sequences, for example in gene therapy, or as vaccines. Many vaccines under development as well as in commercial production are live attenuated viral agents which are used as vectors or a carrier for other viral or bacterial proteins, or other antigens, as vaccine antigens. Live attenuated viral agents including virus like particles, genetically modified or recombinant viral vector (for example recombinant vaccinia, adenoviruses, baculovirus) are included in this invention. These types of vaccines, besides disease prevention, may be used for cancer prevention and therapy. The viral vector vaccine may also be used for gene therapy and drug delivery.

Accordingly the immunogen may be a viral particle or sub-unit, or bacterial cell which acts as a carrier, for example, of a nucleic acid sequence such as a DNA or RNA sequence, in gene therapy, drug delivery, cancer treatment or other purposes. The immunogen may thus not be immunogenic as such, but rather a carrier. Hence, the term “imunogen” as used herein includes an entity capable of provoking an immune response, and an entity which is not necessarily capable of providing an immune response, but rather acts as a carrier or delivery system of, for example, DNA or RNA or protein sequences.

The vaccine composition according to an aspect of this invention is preferably in a free flowing form, powder like form as referred to above. Highly concentrated vaccines may be provided.

Different vaccine powders may be blended together to give multivalent vaccines, free of problems of compatibility (as the vaccines are powders), and with great simplicity and cost effectiveness. Hence, this invention has substantial benefits in the production of multivalent vaccines.

The vaccine composition may be readily dissolved in a pharmaceutically acceptable or veterinarily acceptable diluent, such as buffered saline or other compositions suitable for administration to an animal such as by way of oral administration, subcutaneous administration, administration as an eye drop, aerosol or nasal spray or other mode of administration of vaccines known in the art. Alternatively the vaccine composition may be formed into capsules for administration to a subject orally. Such capsules include, for example, gelatin capsules or other standard capsules used in the pharmaceutical and veterinary fields. Still further, and alternatively, the vaccine composition may be tableted, optionally with standard tabletting excipients and carriers as are well known in the art. In another embodiment the vaccine composition may be coated, for example with an enteric coating which may protect the product from degradation in the stomach and/or to allow for sustained or slow release of the active therefrom.

Free flowing vaccine powders may also be administered transdermally, for example by fine powder administration across the skin as is known in the art, for example using pressurised gases such as hydrogen or helium to move small particles across the skin, using for example PowderJect™ Systems, formerly from PowderJect Pharmaceuticals PLC, (Oxford, United Kingdom), now owned by Chiron Corporation (Emeryville, Calif.).

Throughout this specification and the claims which follow, unless the context requires otherwise, the word “comprise”, or variations such as “comprises” or “comprising”, will be understood to imply the inclusion of a stated integer or step or group of integers or steps but not the exclusion of any other integer or step or group of integers or steps.

The reference to any prior art in this specification is not, and should not be taken as an acknowledgment or any form of suggestion that that prior art forms part of the common general knowledge in Australia.

Non-limiting, illustrative aspects of the present invention will now be described with reference to the following examples.

### EXAMPLE 1

A Huttlin Turbojet spray dryer is modified so as to provide a fluidised bed of particles for contact with a sprayed immunogen containing fluid. In particular, the Turbojet spray dryer was modified to include spray nozzles to provide a fluidized bed, and spray nozzles which spray the immunogen containing fluid from the bottom of the processing vessel in an upward direction.

Commercially available spray drying apparatus spray a solution or slurry into an airstream and allow the material to dry as it falls by gravity. The material may be subsequently sprayed to yield agglomerates. In contrast, in this example, particles of a pharmaceutically acceptable water soluble material are added to the spray dryer so as to provide a fluidised bed. The fluidised bed is sprayed with the immunogen containing fluid.

The dryer was operated at a temperature between about 35° C. and 42° C.

### EXAMPLE 2

Sugar particles, in the form of mannitol or dextrose monohydrate, were loaded into the fluidised bed of Example 1 and fluidised with air at a temperature of 35° C. or 42° C. The fluid air volume was 200 cm/hr.

A commercially available vaccine against avian infectious bronchitis virus, H120 was diluted 1:1 with either:
(a) 5% sucrose, 5% skim milk, purified sterile water to 90%; or
(b) a solution containing gelatin, dextran, phosphate buffer at pH 7, disodium EDTA, mannitol, egg albumin and glycine.

The resulting fluid containing viral particles were then sprayed onto the fluidised sugar core material at a spray rate of 12 g/min per 2 kg batch, at the fluid air volume of 200 cm/hr.

The vaccine composition was recovered from the fluidised bed at a moisture content between 0.1 to 8% as measured using infrared moisture analysis. As an alternative measure of moisture content, the water activity as an endpoint of moisture content can be measured.

Viral infectivity was then measured in chicken embryos by reconstituting the vaccine composition with an equal volume of saline and injecting the vaccine composition into the chicken embryos, and thereafter determining embryo infectious dose EID₅₀. Viral potency was demonstrated for the vaccine composition. The vaccine composition was stable and infective after 7 days storage at ambient room temperature (25° C.) as tested by viral infectivity in chicken embryos.

The H120 virus requires storage at −15° C. to −20° C. and is very temperature sensitive. Thus this example shows vaccine stabilisation.

### EXAMPLE 3

The process of Example 3 was carried out using fluidised mannitol particles, sprayed with a fluid containing the H120 avian infectious bronchitis vaccine mixed with an equal volume of stabilising media (b). The resulting fluid was sprayed onto the fluidised mannitol particles. A free flowing particulate composition was recovered having a moisture level of 2.51%. The process normally took about 20 to 30 minutes to complete.

The same amount of vaccine fluid was subject to freeze drying over a 3 day time period. The end products were then tested for vaccine potency by measuring viral infectivity in chicken embryos according to Example 2. Results are shown in Table 1.

TABLE 1

| Vaccine | Technology | Potency (log EID₅₀) |
|---|---|---|
| ND-H120 | Vaccine Stabilisation Technology | 5.50 |
| □ | (operated at 37° C.) | |
| | Freeze-dried Technology | 5.50 |

The same potency was found in both products.

This example demonstrated that the dried vaccine composition according to the invention had equal potency after production as the freeze dried vaccine. In these tests both compositions were reconstituted with saline and then tested in the chicken embryo model.

The dried composition was recovered after about 30 minutes. This was in direct contrast to the 3 day time period required to produce the freeze dried material.

### EXAMPLE 4

The potency of the vaccine compositions as measure by loss of infectivity on storage at 25° C. or 35° C. was tested in this example.

Vaccine compositions were prepared according to Example 3 using either the H120 vaccine, or a vaccine against avian Infectious Bursal Disease (IBD).

The vaccine compositions were compared with equivalently freeze dried preparations.

In this example, vaccine potency was measured by vaccine infectivity in chicken embryos.

After 7 days storage at 35° C. potency of the vaccine composition of this invention (EID₅₀) had reduced by less than 1 log, giving a highly potent vaccine on storage. In contrast, the freeze dried vaccine had dropped by 3.12 logs for the freeze dried H120 virus and 1.5 logs for the IBD freeze dried vaccine. Stability was also tested on storage at 25° C. After 30 days storage at 25° C., the vaccine composition of this invention for the H120 vaccine dropped by less than 1 log on storage. For the IBD vaccine composition there was 0.85 log reduction in potency after 30 days storage for the vaccine composition of the invention. In contrast using freeze dried IBD vaccine as a comparison, potency dropped by 1.62 logs.

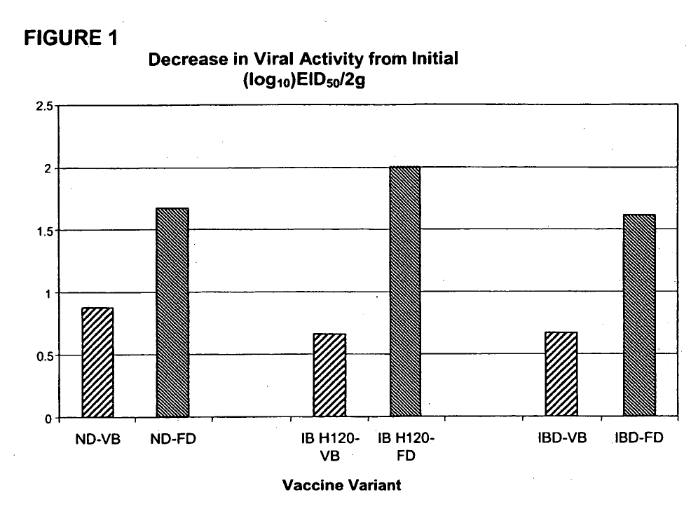

Results are shown in FIG. 1 where IBH120-VB and IBD-VB refer to the vaccine compositions of the invention, respectively containing the avian Infectious Bronchitis H120 vaccine, and avian Infectious Bursal Disease vaccine stored for 30 days at 25° C.

This experiment shows increased stability, as demonstrated by vaccine potency, of the vaccines according to this invention, compared to freeze dried vaccine production.

### EXAMPLE 5

### Newcastle Disease

Vaccine compositions were prepared according to the process of Example 3 using Newcastle disease virus (“ND”, La Sota strain).

The vaccine composition was stored at 25° C. for 30 days, and compared to a freeze-dried sample of the same virus stored under identical conditions. After 30 days, viral activity was assessed in chicken embryos according to Example 2. The compositions of the invention were stable and effective after 30 days, showing a reduction in viral activity of only 0.9 logs. In contrast, freeze-dried compositions showed a decrease in viral activity of 1.7 logs over the same period. These results are show in FIG. 1. FIG. 1 plots decrease in viral activity from initial activity for the compositions of the inventions (ND-VB), and for freeze dried Newcastle disease virus (ND-FD). The first and second shaded columns of this bar graph correspond to this experiment.

Storage at 25° C. for 30 days represents extreme conditions for vaccine storage. Even under these conditions the vaccine compositions of the invention exhibit significant viral activity showing a decrease in infectivity of less than 1 log over 30 days of storage at 25° C.

### EXAMPLE 6

Immunogenicity of the Newcastle disease vaccines of Example 5 was tested in chickens. Four day old SPF chickens were used for the study. Ten chickens were used for each group: ND-VB (Newcastle Disease vaccine according to the invention), ND-FD (freeze dried Newcastle Disease vaccine), and control group which received no vaccine. The EID₅₀/2 g (immunising infective dose) of the freeze dried composition administered was 0.8 logs higher than that of the ND-VB composition. Each chicken received 4 mg of designated vaccine nasally. Their serum samples were collected every 7 days for serology study. Hemagglutination-inhibition (HI) antibody titres in serum were determined using 4HA₅₀ titres of Newcastle virus (La Sota) and 1% chicken erythrocytes solution according to a standard micro method. (Reference: Allen, W. H. and R. E. Gough. “A standard Haemagglutination inhibition test for Newcastle disease. 1. A comparison of macro and micro methods.” Vet. Rec. 95:120-123 (1974).)

Results are shown in Table 2

TABLE 2

| | | | Average HI titres (nlog2) | | | | |
|---|---|---|---|---|---|---|---|
| | EID₅₀/2 g | No of | 7 | 14 | 21 | 28 | 35 |
| Vaccines | (log₁₀) | Chickens | Days | Days | Days | Days | Days |
| ND-VB | 7.38 | 10 | 1.25 | 2.8 | 3.5 | 4.17 | 4.0 |
| ND-FD | 8.17 | 10 | 1.83 | 3.8 | 4.6 | 4.83 | 4.6 |
| None | — | 10 | 0.5 | 0.5 | 0.5 | 0.5 | 0.5 |

Controls showed negligible antibody titres against Newcastle virus. The composition of the invention and freeze dried compositions demonstrated significant specific antibody titre against Newcastle disease virus.

1. A process for the production of a vaccine composition of labile immunogens, wherein a fluid comprising one or more immunogens is sprayed into a reactor containing fluidized particles of a pharmaceutically acceptable water soluble material at a temperature of about 25° C. to about 50° C., such that the immunogen coats and is dried onto the particles under the fluidizing conditions, and thereafter collecting from said reactor dried immunogen containing particles having a moisture content between about 0.1% w/w to about 10% w/w so as to give a stabilized vaccine composition.

2. A process according to claim 1 wherein the immunogen comprises virus particles, bacterial cells or other microorganisms, or antigenic products thereof.

3. A process according to claim 2 wherein the immunogen comprises virus particles or bacterial cells.

4. A process according to claim 2 wherein the immunogen comprises a viral or bacterially derived immunogen selected from a protein, peptide, glycoprotein, or glycolipid, or polysaccharide, optionally associated with a carrier, which on immunization of a subject provokes an immune response to the virus or bacteria from which the immunogen was derived.

5. A process according to claim 1 wherein the fluid comprising one or more immunogens is a viral vaccine or bacterial vaccine preparation mixed with a stabilising diluent to provide a fluid comprising viral particles or bacterial immunogens.

6. A process according to claim 1 wherein the temperature is from about 30° C. to about 46° C.

7. A process according to claim 1 wherein the moisture content is from 0.1% w/w to 2.6% w/w.

8. A process according to claim 7 wherein the moisture content is from 0.2% w/w to 1.5% w/w.

9. A process according to claim 1 wherein said fluid comprising one or more immunogens is a suspension of dispersion of immunogens selected from viral particles, bacterial cells or other microorganisms, eukaryotic cells, or antigenic products of said immunogens.

10. A process according to claim 1 wherein said fluid containing one or more immunogens includes one or more amino acids, proteins, chelating agents, buffers, preservatives, stabilizers, mineral salts, metal antioxidants, lubricants and adjuvants.

11. A process according to claim 9 wherein viral particles or bacterial cells in a culture medium, vaccine composition or other fluid are diluted with a diluent.

12. A process according to claim 1 wherein said particles of a pharmaceutically acceptable water soluble material comprise one or more monosaccharide, disaccharide, polysaccharide, carbohydrate, water soluble peptide, mineral salt, water soluble polymer, or water soluble pharmaceutically acceptable excipient.

13. A process according to claim 1 wherein said pharmaceutically acceptable water soluble material comprises one or more sugars.

14. A process according to claim 1 wherein the pharmaceutically acceptable water soluble material comprises a particle size from 20 microns to 1 mm.

15. A process according to claim 14 wherein said particle size is from 50 microns to 200 microns.

16. A process according to claim 1 wherein said reactor is a spray drying reactor of fluidized bed into which immunogen containing fluid is sprayed onto fluidized particles and dried thereon.

17. A process according to claim 16 wherein fluid comprising one or more immunogens is sprayed through a nozzle or spray head which delivers the sprayed fluid into the reactor.

18. A process according to claim 16 wherein said particles are fluidized in a reactor containing a fluidized bed at a rate between 200 to 500 m²/h.

19. A process according to claim 1 wherein said stabilized vaccine composition is stable and efficacious on storage at 25° C. for 30 days.

20. A process according to claim 1 wherein the vaccine composition is a free flowing particulate material.

21. A process according to claim 1 which further comprises mixing together two or more free flowing stabilized vaccine compositions containing different immunogens to give a multivalent vaccine composition.

22. A process according to claim 3 wherein said virus particles or bacteria is a carrier for the delivery of DNA sequences, RNA sequences or vaccine antigens.

23. A process according to claim 3 wherein said virus particles or bacteria are genetically modified.

24. A stabilized vaccine composition comprising immunogen coated particles of a pharmaceutically acceptable water soluble material, the composition having a moisture content between about 0.1% w/w to about 10% w/w.

25. A vaccine composition according to claim 24 wherein the immunogen comprises virus particles, bacterial cells or other microorganisms or antigenic products thereof.

26. A vaccine composition according to claim 24 wherein the immunogen comprises virus particles or bacterial cells.

27. A vaccine composition according to claim 26 which contains live virus particles capable of reproduction in an immunized host.

28. A vaccine composition according to claim 24 wherein the immunogen comprises a viral or bacterially derived immunogen selected from a protein, peptide, glycoprotein, or glycolipid, or polysaccharide, optionally associated with a carrier, which on immunization of a subject provokes an immune response to the virus or bacteria from which the immunogen was derived.

29. A vaccine composition according to claim 24 which is stable and efficacious on storage at 25° C. for 30 days.

30. A vaccine composition according to claim 24 wherein the pharmaceutically acceptable water soluble material comprises one or more of a monosaccharide, disaccharide, polysaccharide or carbohydrate, water soluble peptide or peptides, gelatine, mineral salt or water soluble polymer, or water soluble pharmaceutically acceptable excipient.

31. A vaccine composition according to claim 30 wherein said water soluble material comprises one or more sugars.

32. A vaccine composition according to claim 24 comprising two or more different immunogen coated particles, so as to give a multivalent vaccine.

33. A vaccine composition according to claim 24 wherein the immunogen is a carrier of a nucleic acid sequence or a peptide or polypeptide.

34. A vaccine composition according to claim 24 which comprises a particle size from 50 microns to 400 microns.

35. A process according to claim 34 wherein said particle size is from 50 microns to 200 microns.

36. A composition according to claim 24 wherein said immunogen coated particles include one or more amino acids, proteins, chelating agents, buffers, preservatives, stabilizers, mineral salts, antioxidants, lubricants and adjuvants.

37. A vaccine composition according to claim 24 which is a free flowing particulate composition.

38. A vaccine composition according to claim 24 which is immunogenic on administration to an animal or human.

39. A vaccine composition according to claim 24 which is a human or animal vaccine.

40. A vaccine according to claim 39 which is a poultry vaccine for the prevention of Newcastle Disease, infectious bronchitis, coccidiosis, fowl pox, fowl cholera, reovirus induced tenosynovitis (viral arthritis), fowl laryngotracheitis, avian encephalomyelitis, infectious bursal disease (IBD), Marek's Disease, salmonella infection, mycoplasma gallisepticum infection, avian rhinotracheitis, avian herpes and Mycoplasma hyponeumoniae, Egg Drop Syndrome, Infectious Coryza (Haemophilis pasagallinarum), mycoplasma synoviae or avian reovirus.

41. A vaccine composition according to claim 39 is a porcine vaccine, for the prevention or treatment of Actinobacillus pleuropneumoniae, atrophic rhinitis, pseudorabies, swine erysipelas, porcine parvovirus, E-coli enterotoxicosis, myoplasma hyopneumoniae, influenza, leptospira, E.-coli infection, Porcine Reproductive and Respiratory Syndrome (PRRS), Bordetella and multocida types A and D infections, haemophilus parasuis infection, clostridium perfringens infection, rotavirus infection, Streptococcus suis infection, Glasser's Disease, pneumonia, bordetella bronchiseptica infection.

42. A vaccine according to claim 39 which is a human vaccine for the prevention of influenza, hepatitis A, hepatitis B, hepatitis C, herpes simplex virus (type 2), polio, diphtheria, pertussis, haemophilus influenza type B (Hib), measles, mumps, rubella, typhoid fever, varicella (chicken pox), Dengue fever, Epstein-Barr virus infection, human papillomavirus infection, Streptococcus pnemoniae infection, Neisseria meningitidis infection, Pneumococcal infection, viral meningitis, rotavirus infection, tick-borne encephalitis, travel diarrhea, cholera, yellow fever or tuberculosis.
